SEQUENCE LISTING (4 sequences)

SEQ ID NO: 1 - DNA, 2703 bp, homo sapiens
  CDS (640)..(2142): GDF-5 precursor
ccatggcctcgaaagggcagcggtgatttttttcacataaatatatcgcacttaaatgagtttagacagcatgacatcagagagtaattaaattggtttgggttggaattccgtttccaattcctgagttcaggtttgtaaaagatttttctgagcacctgcaggcctgtgagtgtgtgtgtgtgtgtgtgtgtgtgtgtgtgtgtgtgaagtattttcactggaaaggattcaaaactagggggaaaaaaaaactggagcacacaggcagcattacgccattcttccttcttggaaaaatccctcagccttatacaagcctccttcaagccctcagtcagttgtgcaggagaaagggggcggttggctttctcctttcaagaacgagttattttcagctgctgactggagacggtgcacgtctggatacgagagcatttccactatgggactggatacaaacacacacccggcagacttcaagagtctcagactgaggagaaagcctttccttctgctgctactgctgctgccgctgcttttgaaagtccactcctttcatggtttttcctgccaaaccagaggcacctttgctgctgccgctgttctctttggtgtcattcagcggctggccagaggatgagactccccaaactcctcactttcttgctttggtacctggcttggctggacctggaattcatctgcactgtgttgggtgcccctgacttgggccagagaccccaggggaccaggccaggattggccaaagcagaggccaaggagaggccccccctggcccggaacgtcttcaggccagggggtcacagctatggtgggggggccaccaatgccaatgccagggcaaagggaggcaccgggcagacaggaggcctgacacagcccaagaaggatgaacccaaaaagctgccccccagaccgggcggccctgaacccaagccaggacaccctccccaaacaaggcaggctacagcccggactgtgaccccaaaaggacagcttcccggaggcaaggcacccccaaaagcaggatctgtccccagctccttcctgctgaagaaggccagggagcccgggcccccacgagagcccaaggagccgtttcgcccaccccccatcacaccccacgagtacatgctctcgctgtacaggacgctgtccgatgctgacagaaagggaggcaacagcagcgtgaagttggaggctggcctggccaacaccatcaccagctttattgacaaagggcaagatgaccgaggtcccgtggtcaggaagcagaggtacgtgtttgacattagtgccctggagaaggatgggctgctgggggccgagctgcggatcttgcggaagaagccctcggacacggccaagccagcggcccccggaggcgggcgggctgcccagctgaagctgtccagctgccccagcggccggcagccggcctccttgctggatgtgcgctccgtgccaggcctggacggatctggctgggaggtgttcgacatctggaagctcttccgaaactttaagaactcggcccagctgtgcctggagctggaggcctgggaacggggcagggccgtggacctccgtggcctgggcttcgaccgcgccgcccggcaggtccacgagaaggccctgttcctggtgtttggccgcaccaagaaacgggacctgttctttaatgagattaaggcccgctctggccaggacgataagaccgtgtatgagtacctgttcagccagcggcgaaaacggcgggccccactggccactcgccagggcaagcgacccagcaagaaccttaaggctcgctgcagtcggaaggcactgcatgtcaacttcaaggacatgggctgggacgactggatcatcgcaccccttgagtacgaggctttccactgcgaggggctgtgcgagttcccattgcgctcccacctggagcccacgaatcatgcagtcatccagaccctgatgaactccatggaccccgagtccacaccacccacctgctgtgtgcccacgcggctgagtcccatcagcatcctcttcattgactctgccaacaacgtggtgtataagcagtatgaggacatggtcgtggagtcgtgtggctgcaggtagcagcactggccctctgtcttcctgggtggcacatcccaagagccccttcctgcactcctggaatcacagaggggtcaggaagctgtggcaggagcatctacacagcttgggtgaaaggggattccaataagcttgctcgctctctgagtgtgacttgggctaaaggcccccttttatccacaagttcccctggctgaggattgctgcccgtctgctgatgtgaccagtggcaggcacaggtccagggagacagactctgaatgggactgagtcccaggaaacagtgctttccgatgagactcagcccaccatttctcctcacctgggccttctcagcctctggactctcctaagcacctctcaggagagccacaggtgccactgcctcctcaaatcacatttgtgcctggtgacttcctgtccctgggacagttgagaagctgactgggcaagagtgggagagaagaggagagggcttggatagagttgaggagtgtgaggctgttagactgttagatttaaatgtatattgatgagataaaaagcaaaactgtgcct

SEQ ID NO: 2 - protein, 501 aa, homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 3 - DNA, 360 bp, Homo sapiens
  CDS (1)..(360): human mature monomeric GDF-5
gcaccactagcaactcgtcagggcaagcgacccagcaagaaccttaaggctcgctgcagtcggaaggcactgcatgtcaacttcaaggacatgggctgggacgactggatcatcgcaccccttgagtacgaggctttccactgcgaggggctgtgcgagttcccattgcgctcccacctggagcccacgaatcatgcagtcatccagaccctgatgaactccatggaccccgagtccacaccacccaccgcctgtgtgcccacgcgactgagtcccatcagcatcctcttcattgactctgccaacaacgtggtgtataagcagtatgaggacatggtcgtggagtcgtgtggctgtagg

SEQ ID NO: 4 - protein, 120 aa, Homo sapiens
APLATRQGKRPSKNLKARCSRKALHVNFKDMGWDDWIIAPLEYEAFHCEGLCEFPLRSHLEPTNHAVIQTLMNSMDPESTPPTACVPTRLSPISILFIDSANNVVYKQYEDMVVESCGCR

(19) United States
(12) Patent Application Publication
(10) Pub. No.: US 2016/0317666 A1
(43) Pub. Date: Nov. 3, 2016

(54) DRUG DELIVERY DEVICES AND GROWTH FACTOR FORMULATIONS FOR ACCELERATED WOUND HEALING

(71) Applicant: Biopharm Gesellschaft zur biotechnologischen Entwicklung von Pharmaka mbH, Heidelberg (DE)

(72) Inventor: Frank PLOEGER, Heidelberg (DE)

(73) Assignee: Biopharm Gesellschaft zur biotechnologischen Entwicklung von Pharmaka mbH, Heidelberg (DE)

(21) Appl. No.: 15/089,862

(22) Filed: Apr. 4, 2016

Related U.S. Application Data
(62) Division of application No. 13/810,961, filed on Jan. 18, 2013.

(30) Foreign Application Priority Data
Jul. 30, 2010 (EP) 10171488.9

(51) Int. Cl. A61K 47/26; A61K 38/18

(52) U.S. Cl. CPC A61K 47/26; A61K 38/1875

(57) ABSTRACT

The present invention is directed to novel drug delivery devices and pharmaceutical compositions containing growth and differentiation factor proteins. Said devices and compositions are specifically designed to accelerate tissue regeneration and wound healing processes of mammalian tissues. The invention is especially useful for the supportive therapy of diabetic wounds and ulcers.

This application is a divisional of U.S. Ser. No. 13/810,961, filed Jan. 18, 2013, which is a 35 U.S.C. 371 National Phase Entry Application from PCT/EP2011/063128, filed Jul. 29, 2011, which claims the benefit of European Patent Application No. 10171488.9 filed on Jul. 30, 2010, the disclosures of which are incorporated herein in their entirety by reference.

### DESCRIPTION

The present invention is directed to novel drug delivery devices and pharmaceutical compositions containing growth and differentiation factor proteins. Said devices and compositions are specifically designed to accelerate tissue regeneration and wound healing processes of mammalian tissues. The invention is especially useful for the supportive therapy of diabetic wounds and ulcers.

GDF-5 (Hötten et al. 1994, Biochem. Biophys Res. Commun. 204, 646-652) is a morphogen which has been shown to promote cell proliferation, differentiation and/or tissue formation in several tissues. The protein is also known as morphogenic protein MP52, bone morphogenetic protein-14 (BMP-14) or cartilage-derived morphogenetic protein-1 (CDMP-1). GDF-5 is closely related to GDF-6 and GDF-7. These three proteins form a distinct subgroup of the TGF-β superfamily, thus displaying comparable biological properties and an extraordinary high degree of amino acid sequence identity (see i.e. Wolfman et al. 1997, J. Clin. Invest. 100, 321-330). All family members are initially synthesized as larger precursor proteins which subsequently undergo proteolytic cleavage at a cluster of basic residues approximately 110-140 amino acids from the C-terminus, thus releasing the C-terminal mature protein parts from the N-terminal prodomain. The mature polypeptides are structurally related and contain a conserved bioactive domain comprising six or seven canonical cysteine residues which is responsible for the characteristical three-dimensional “cystine-knot” motif of these proteins. Native GDF-5 related proteins are homodimeric molecules and act mainly through interaction with specific receptor complexes which are composed of type I and type II serine/threonine receptor kinases. The receptor kinases subsequently activate Smad proteins, which then propagate the signals into the nucleus to regulate target gene expression.

It has repeatedly been demonstrated that members of the GDF-5/-6/-7 subgroup are primarily important inducers and regulators of bone and cartilage (Cheng et al. 2003, J. Bone & Joint Surg. 85A, 1544-1552; Settle et al. 2003, Developm. Biol. 254, 116-130). GDF-5 is a natural growth factor in the nervous system (see for example WO 97/03188; Krieglstein et al., (1995) J. Neurosci Res. 42, 724-732; Sullivan et al., (1997) Neurosci Lett 233, 73-76; Sullivan et al. (1998), Eur. J. Neurosci 10, 3681-3688). Furthermore, it is e.g. useful for the modulation of skin related tissue growth (WO 02/076494; Battaglia et al. 2002, Trans. Orthop. Res. Soc. 27, 584), and for induction of angiogenic processes (Yamashita et al. 1997, Exp. Cell Res. 235, 218-26).

After discovery of their unique tissue inductive activities, growth factor proteins such as GDF-5 have been successfully applied in therapeutic research and regenerative surgery, in which they promote and assist the natural healing process of various damaged tissues, either alone or in combination with specific matrix materials. Although several pharmaceutical compositions comprising biologically active mature GDF-5 related proteins have been developed (see e.g. WO96/33215), formulation and handling of GDF-5 are nevertheless still problematic because the mature protein tends to interact with a couple of solid materials and shows exceptional poor solubility under physiological conditions. A pH-dependent solubility profile of mature GDF-5/MP52 (shown i.e. in EP 1 462 126) reveals that the protein starts precipitating in aqueous solutions with a pH above 4.25 and becomes almost unsoluble between pH 5 and pH 9.

According to these facts, previous attempts to formulate stable liquid or gel-like GDF-5 compositions have faced serious problems. Limited success was achieved for special applications only. EP 1 462 126 which describes materials for bone regeneration succeeded in improving the protein solubility profile slightly by using solvents with low ionic strength. WO2008/049588 describes a lipid nanosphere formulation which is capable of delivering significant quantities of GDF-5 related proteins in vivo along neuronal pathways. However, the combination of GDF-5 related proteins with particular materials remains challenging. Nevertheless there is still a great need to develop novel methods and pharmaceutical compositions for the efficient administration of such proteins under physiological conditions.

For wound healing purposes, both lotion-like and solid surgical dressings of various forms, sizes and materials have been developed which are primarily designed to ensure wound closure under semi-sterile conditions. Several of these dressings are made up of organic materials such as e.g. collagens whereas other devices are composed of synthetic components such as e.g. amorphous thermoplastic polymers. Some wound dressings of the most advanced generation feature additional drug delivery functions; they are capable of administering bioactive substances such as antibiotics or cytokines like epidermal growth factor (EGF) or platelet-derived growth factor (PDGF/Becaplermin). For example, genetically engineered PDGF is commercially available under the brand name Regranex® as a topical (0.01%) wound healing gel which has been approved for the treatment of diabetic foot ulcers that extend into the subcutaneous tissue or beyond.

Especially desirable for wound healing and other tissue regeneration purposes are new formulations, surgical dressings and drug delivery devices through which bioactive substances are being delivered to the human body in a controlled manner, thus precisely satisfying the high demand of the body. It is therefore an object of the invention to improve the therapeutic usability of GDF-5 and related proteins by providing stable and non-toxic growth factor compositions which are applicable in combination with synthetic wound healing materials and devices described hereinafter.

Definitions:

In order to avoid misunderstandings and ambiguities, some frequently used terms herein are defined and exemplified as follows:

The term “cystine-knot domain” as used herein means the well known and conserved cysteine-rich amino acid region which is present in the mature parts of TGF-beta superfamily proteins such as i.e. human GDF-5 and forms a three-dimensional protein structure known as cystine-knot. In this domain the respective location of the cysteine residues to each other is important and is only allowed to vary slightly in order not to lose the biological activity. It has been demonstrated that the cystine-knot domain alone is sufficient for the biological function of the protein (Schreuder et al. (2005), Biochem Biophys Res Commun. 329, 1076-86). Consensus sequences for cystine-knot domains are well known in the state of the art. According to the definition defined herein the cystine-knot-domain of a protein starts with the first cysteine residue participating in the cystine-knot of the respective protein and ends with the residue which follows the last cysteine participating in the cystine-knot of the respective protein. For example, the cystine-knot domain of the human GDF-5 precursor protein (SEQ ID NO: 2) consists of the amino acids 400-501 (see also FIG. 1).

The term “GDF-5-related protein” as used herein means any naturally occurring or artificially created protein which comprises a cystine-knot-domain with an amino acid identity of at least 60% to the 102 aa cystine-knot domain of human GDF-5 (amino acids 400-501 of SEQ ID NO: 2). This term includes proteins belonging to the group of GDF-5, GDF-6 and GDF-7 proteins from vertebrate or mammalian species as well as recombinant variants thereof as long as these proteins show the above mentioned percentage of identity with the cystine-knot domain of human GDF-5. The limiting value of 60% is well suitable to separate members of the GDF-5/-6/-7 group of proteins as well as variants thereof from further proteins such as other GDFs and BMPs. A comparison of the 102 aa cystine-knot-domains of human GDF-5, human GDF-6 and human GDF-7 (see FIG. 2) reveals the high grade of amino acid identity between these proteins. Human GDF-6 shares 87 (85%) and human GDF-7 shares 83 (81%) identical residues with the cystine-knot-domain of human GDF-5. The respective domains of GDF-5/-6/-7 molecules from other vertebrate and mammalian species which have been identified so far also show very high identity percentages of at least 75% (between 79% and 99%), when compared with human GDF-5. In contrast, GDFs and BMPs not belonging to the GDF-5/-6/-7 subgroup display much lower identity values below 60% (see FIG. 3)

The determination of corresponding amino acid positions in related amino acid sequences as well as the calculation of percentages of identity between can be easily performed with the help of well known alignment algorithms and optionally computer programs using these algorithms. For example, the amino acid identities in this patent application (i.e. FIG. 2) have been calculated by aligning sequences with the freeware program ClustalX (Version 1.81) with default parameters and subsequent counting of identical residues by hand. Default settings for pairwise alignment (slow-accurate) are: gap opening parameter: 10.00; gap extension parameter 0.10; Protein weight matrix: Gonnet 250. The ClustalX program is described in detail in Thompson, J. D., Gibson, T. J., Plewniak, F., Jeanmougin, F. and Higgins, D. G. (1997): The ClustalX windows interface: flexible strategies for multiple sequence alignment aided by quality analysis tools. Nucleic Acids Research 24:4876-4882. ClustalX is a windows interface for the ClustalW multiple sequence alignment program and is i.e. available from various sources, i.e. by anonymous ftp from ftp-igbmc.u-strasbg.fr, ftp.embl-heidelberg.de, ftp.ebi.ac.uk or via download from the following webpage: http://www-igbmc.u-strasbg.fr/Biolnfo/. The ClustalW program and algorithm is also described in detail in Thompson, J. D., Higgins, D. G. and Gibson, T. J. (1994): CLUSTALW: improving the sensitivity of progressive multiple sequence alignment through sequence weighting, positions-specific gap penalties and weight matrix choice. Nucleic Acids Research 22:4673-4680.

The term “variant” as used herein means any of the following polypeptides:

a) biologically active fragments of a protein, preferably at least comprising the cystine-knot domain;

b) biologically active protein constructs which contain additional sequences either with or without adding biological functions) in excess to the original sequence of the protein or constructs which contain amino acid substitutions;

c) any combination of a) and b).

The term “medical device” refers to any material (single or composite), instrument or apparatus or, whether used alone or in combination, is intended to be used by the manufacturer for the purpose of treatment or alleviation of disease or injury. This includes devices that do not achieve its principal intended action in or on the human body by pharmacological, immunological or metabolic means, but which may be assisted in its function by such means.

The term “amorphous thermoplastic polymers” refers to synthetic polymeric materials having a randomly ordered molecular structure and which can be formed into a load-bearing shape. For better understanding: high temperature materials are divided into two main categories—semi-crystalline and amorphous—based on their difference in molecular structure. Solid organic compounds consisting of ordinary small molecules tend to be crystalline, that is, the molecules pack themselves in regular three-dimensional arrays. Polymers are different; they can be amorphous (totally lacking positional order on the molecular scale, thereby creating local regions with different charge) or semi-crystalline (containing varying percentages of crystalline regions in the same sample). Amorphous thermoplastic polymers have characteristic attributes: they do not have a sharp melt point; they change viscosity when heated and they are isotropic in flow. As a result, amorphous materials typically exhibit lower mold shrinkage and fewer tendencies to warp than the semi-crystalline materials. The term “amorphous thermoplastic polymers” comprises polymeric materials such as polysulfones (PSU), polyethersulfones (PES), polyphenylsulfones (PPSU), polystyrenes, polyetherimides, polyarylates, polyvinylchlorides, polyamides (amorphous), polymethylmethacrylates, polyamideimides, and acrylonitrile butadiene styrenes.

The term “polysulfone-based plastics” denotes a group of thermoplastic polymers containing the subunit aryl-SO₂-aryl, the defining feature of which is the sulfone group. The repeating unit of these polymers is shown in FIG. 5. The material allows easy manufacturing of membranes, with reproducible properties and controllable size of pores down to 40 nanometres. Polysulfone-based plastics can be reinforced with glass fibers and are also used as a copolymer. The group of “polysulfone-based plastics” contains the chemically related substances polysulfone (also spelled polysulphone, abbreviated PSU), polyethersulfone (also spelled polyethersulphone, abbreviated PES), and polyphenylsulfone (also spelled polyphenylsulphone, abbreviated PPSU).

The term “polysulfones” (PSU) denotes thermoplastic polymers (CAS identifier 25154-01-2) with the repeating unit shown in FIG. 6.

The term “polyethersulfones” (PES) denotes thermoplastic polymers (CAS identifier 113569-14-5) with the chemical formula Poly(oxy-1,4-phenylsulfonyl-1,4-phenyl). The substance is closely related to polysulfones and polyphenylsulfones. A repeating unit of PES is shown in FIG. 7

The term “polyphenylsulfones” (PPSU) denotes thermoplastic polymers (CAS identifier 25608-64-4) with the repeating unit shown in FIG. 8. The substance is closely related to polysulfones and polyethersulfones.

The term “polyetherimides” (PEI) denotes thermoplastic polymers (CAS identifier 61128-46-9) with the repeating unit shown in FIG. 9. The molecular formula of repeating unit of PEI is C₃₇H₂₄O₆N₂ and the molecular weight is 592 g/mol.

The term “carboxymethyldextran” (CMD) denotes polyanionic derivatives of dextran with varying amounts of carboxymethyl groups attached by a stable O-ether linkage. CMDs of various molecular weight (common molecular weights of CMD are e.g. 10 000, 40 000, 1 000 000) are commercially available. According to this definition and the invention disclosed hereinafter, the CMD might be also further chemically modified (e.g. by carrying additional binding groups), as long as the molecule is still capable of preventing the molecular stickiness between GDF-5 related proteins and the solid materials described in this patent application, e.g. amorphous thermoplastic polymers, polyvinylpyrrolidon or biodegradable materials such as e.g. woven or nonwoven collagen, gelatine, polylactide (PLA), polyglycolide (PGA), polycaprolactone (PCL) polylactid, dextran, hyaluronic acid and chitosan or combinations thereof.

The term “biological activity” denotes the activity of compounds, including, e.g., a GDF-5-related protein as measured by the common in vitro alkaline phosphatase assay (ALP), e.g. as described in example 8 or in Takuwa et al. (1989), Am. J. Physiol. 257, E797-E803). Suitable cell lines which may be used in such ALP assay are e.g. ATDC-5 or MCHT 1/26 cells.

In brief, the present invention describes a significant improvement of the medical device-mediated delivery of bioactive GDF-5 related proteins towards sites where tissue regeneration is desirable. More precisely, the invention enables the use and delivery of these growth factor proteins in combination with medical devices made of certain synthetic and natural materials.

GDF-5 and the closely related proteins GDF-6 and GDF-7 feature an uncommon surface charge distribution pattern. In contrast to most other hydrophobic proteins, the surface of GDF-5 is not predominantly composed of hydrophobic amino acids such as alanine, valine and leucine and features an uncommon charge distribution effect which is shown in FIG. 4. Over a wide pH range, the surface of GDF-5 comprises large regions of varying length with opposite charge. These protein parts attract each other and thereby seem to initiate coagulation and precipitation of the growth factor molecule. The uncommon charge distribution of GDF-5 related proteins also increases the adhesion to several solid materials with alternating charge patterns or other uncommon substance characteristics.

The adhesion between GDF-5 related proteins and solid materials is e.g. noticeable in case of polyvinylpyrrolidon and certain biodegradable materials such as e.g. woven or nonwoven collagen, gelatine, polylactide (PLA), polyglycolide (PGA), polycaprolactone (PCL) polylactid, dextran, hyaluronic acid and chitosan or combinations thereof. However, said unwanted adhesion effect is particularly strong if certain amorphous thermoplastic polymers are used in combination with GDF-5, for example if they are part of a medical drug delivery device. Due to their amorphous character with the polymeric string not regularly folded but disordered, these synthetic polymers—although consisting of high numbers of identical repeating units (see e.g. FIGS. 5 to 9)—exhibit a randomly ordered molecular structure with both hydrophobic and more polar/hydrophilic regions. There is evidence that inter alia this attribute, in combination with further topographical features, causes the strong molecular stickiness in case of a direct contact with a solution comprising GDF-5 related proteins.

According to the present invention, said molecular stickiness between GDF-5 related proteins and various materials, particularly amorphous thermoplastic polymers which are preferably selected from the group consisting of polysulfone-based plastics and polyetherimides and which are most preferably selected from the group consisting of polysulfones (PSU), polyethersulfones (PES) and polyphenylsulfones (PPSU) can be efficiently prevented with the help of certain organic additives belonging to the group of carbohydrates.

In a first embodiment of the invention, said organic additive is trehalose. Trehalose, also known as mycose or tremalose, is a natural alpha-linked disaccharide formed by an α,α-1,1-glucoside bond between two α-glucose units. More precisely, addition of a defined amount of trehalose to a solution of one or more GDF-5 related proteins causes an efficient blocking of adhesional interactions between GDF-5 related proteins and amorphous thermoplastic polymers. According to the experiments performed by the inventor, it is required for a sufficient blocking effect that the solution of one or more GDF-5 related proteins contains between 0.001 and 5% trehalose. Particularly good effects could be observed with a solution containing between 0.01 and 3% trehalose and even better effects with a solution containing between 0.1 and 2% trehalose. Best effects were seen if said solution contained between 0.5 and 1% trehalose.

In a second embodiment of the invention, said organic additive is dextran sulphate or carboxymethyldextran sulfate, which can be used in most cases as a replacement for trehalose. Dextran sulfate is a long-chain (with variable length) polymer of glucose containing 17-20% sulphur. It is preferred that a dextran sulfate with an average molecular weight between 100 and 10 000, most preferably 500 (e.g. Amersham 17-0340-01), is used. Good blocking effects were seen if the protein solution contained between 0.001 and 5% dextran sulfate or carboxymethyldextran sulfate, preferably between 0.01 and 3% dextran sulfate or carboxymethyldextran sulfate, more preferably between 0.1 and 2% dextran sulfate or carboxymethyldextran sulfate and most preferably between 0.5 and 1% dextran sulfate or carboxymethyldextran sulfate.

In a third embodiment of the invention, said organic additive is carboxymethyl dextran which is chemically closely related to carboxymethyldextran sulphate. Good blocking effects were seen if the protein solution contained between 0.001 and 5% carboxymethyl dextran, preferably between 0.01 and 3% carboxymethyl dextran, more preferably between 0.1 and 2% carboxymethyl dextran and most preferably between 0.5 and 1% carboxymethyl dextran.

As e.g. shown in example 4, even very low concentrations (0.001%) of either trehalose or dextran sulfate prevented the binding of at least 75% of rhGDF-5 to hollow fibre capillaries made of PES or other amorphous thermoplastic polymers. With higher carbohydrate concentrations, nearly 100% of the injected rhGDF-5 could be detected after hollow fibre capillary passage. Thus, in certain embodiments of the invention, the concentration of trehalose, dextran sulfate, carboxymethyl dextran sulfate or carboxymethyl dextran prevents the binding of at least 75%, at least 85%, at least 90%, at least 95% or at least 99% of dissolved GDF-5 related proteins to amorphous thermoplastic polymers.

According to the invention, the apparatus or medical device which has a storage and/or delivery function of GDF-5 related proteins and which comprises one or more amorphous thermoplastic polymers may have different forms, shapes, styles or designs. For example, the medical drug delivery device shown in example 9 is a hollow fibre capillary device made of polyethersulfone (PES). Generally preferred are medical devices containing one or more structural elements for the storage or delivery of GDF-5 related proteins which are selected from the group consisting of a hollow fibre, a capillary, a tubing, a tank, a container, a mesh, a spongy element, and/or a membrane.

The parts and embodiments of the invention described in this patent specification apply to all naturally occurring or artificially designed proteins which are very closely related to human growth/differentiation factor 5 (hGDF-5). The term “GDF-5-related proteins” includes functionally similar proteins belonging to the group of vertebrate GDF-5, GDF-6 and GDF-7 proteins as well as recombinant variants thereof. Due to an extraordinary high degree of amino acid sequence identity (see FIG. 2), this group of proteins exhibits comparable biological properties. Common feature of all GDF-5-related proteins is the occurrence of a bioactive cystine-knot-domain with an amino acid identity of at least 60% to the 102 aa cystine-knot domain of human GDF-5/SEQ ID NO: 2 which is sufficient for the biological function of the protein. As can be seen from FIG. 3, the preferred limiting value of 60% separates members of the GDF-5/-6/-7 group from more distantly related GDFs and BMPs. Especially preferred proteins display amino acid identities of at least 70%, 80% or 90% to the 102 aa cystine-knot domain of human GDF-5.

Non-limiting examples for vertebrate and mammalian GDF-5-related proteins are precursors and mature proteins of human GDF-5 (disclosed as MP52 in WO95/04819 and as human GDF-5 in Hötten et al. 1994, Biochem. Biophys Res. Commun. 204, 646-652), recombinant human (rh) GDF-5/MP52 (WO96/33215), MP52 Arg (WO97/06254); HMW human MP52s (WO97/04095), CDMP-1 (WO96/14335), mouse (Mus musculus) GDF-5 (U.S. Pat. No. 5,801,014), rabbit (Oryctolagus cuniculus) GDF-5 (Sanyal et al. 2000, Mol Biotechnol. 16, 203-210), chicken (Gallus gallus) GDF-5 (NCBI accession no. NP_989669), african clawed frog (Xenopus laevis) GDF-5 (NCBI accession no. AAT99303), monomeric GDF-5 (WO 01/11041 and WO 99/61611), human GDF-6/BMP-13 (U.S. Pat. No. 5,658,882), mouse GDF-6 (NCBI accession no NP_038554), GDF-6/CDMP-2 (WO96/14335), human GDF-7/BMP-12 (U.S. Pat. No. 5,658,882), mouse GDF-7 (NCBI accession no AAP97721), GDF-7/CDMP-3 (WO96/143335). Covered by the invention are also GDF-5-related proteins having additional mutations such as substitutions, additions and deletions, as long as these additional mutations do not completely abolish the biological protein activity. Some preferred variants are mutants of GDF-5-related proteins with improved biological activity. For example, one or more residues which are normally present in the human GDF-5 precursor protein (see FIG. 1) are substituted in these mutants by other amino acids: the arginine at position 438 of the human GDF-5 precursor is replaced by glycine, alanine, valine, leucine, isoleucine, methionine or asparagines; and/or serine 439 is replaced by aspartic acid, glutamic acid, glycine, leucine, or isoleucine; and/or asparagine 445 is replaced by serine or threonine. In another high activity mutant, methionine 453 and/or methionine 456 are replaced by alanine, valine, or isoleucine. Also of special interest are mutants in which leucine 441 is replaced by proline.

In general, the present invention can be applied in all situations in which storage and/or delivery of the above mentioned recombinant and wild-type GDF-5 forms in combination with medical devices made of synthetic polymeric compounds are useful. Thus, the present invention can be used to facilitate the regeneration of various tissues and organs. For example, GDF-5 is considered to be a very effective promoter of bone and cartilage formation as well as connective tissue formation (see for example WO 95/04819, Hötten et al. 1996, Growth Factors 13, 65-74; Storm et al. 1994, Nature 368, 639-643; Chang et al. 1994, J. Biol. Chem. 269, 28227-28234) and formation of connective tissue attachment (EP 0 831 884). In this context, GDF-5 is useful for applications concerning the joints between skeletal elements (see for example Storm & Kingsley 1996, Development 122, 3969-3979). One example for connective tissue is tendon and ligament (Wolfman et al. 1997, J. Clin. Invest. 100, 321-330; Aspenberg & Forslund 1999, Acta Orthop Scand 70, 51-54; WO 95/16035). The protein is helpful for meniscus and spinal/intervertebral disk repair (Walsh et al. 2004, Spine 29, 156-63) and spinal fusion applications (Spiro et al. 2000, Biochem Soc Trans. 28, 362-368). GDF-5 can be beneficially applied in tooth (dental and periodontal) applications (see for example WO 95/04819; WO 93/16099; Morotome et al. 1998, Biochem Biophys Res Comm 244, 85-90) such as the regeneration of dentin or periodontal ligament. GDF-5 is also useful in wound repair of any kind. It is also beneficial for promoting tissue growth in the neuronal system and survival of e.g. dopaminergic neurons. In this context, GDF-5 can be used for treating neurodegenerative disorders like e.g. Parkinson's disease and possibly also Alzheimer's disease or Huntington chorea tissues (see for example WO 97/03188; Krieglstein et al., (1995) J. Neurosci Res. 42, 724-732; Sullivan et al., (1997) Neurosci Lett 233, 73-76; Sullivan et al. (1998), Eur. J. Neurosci 10, 3681-3688). GDF-5 allows to maintain nervous function or to retain nervous function in already damaged tissues. GDF-5 is therefore considered to be a generally applicable neurotrophic factor. It is also useful for diseases of the eye, in particular retina, cornea and optic nerve (see for example WO 97/03188; You et al. (1999), Invest Opthalmol V is Sci 40, 296-311), for hair growth and the treatment and diagnosis of skin related disorders (WO 02/076494; Battaglia et al. 2002, Trans. Orthop. Res. Soc. 27, 584), and for induction of angiogenesis (Yamashita et al. 1997, Exp. Cell Res. 235, 218-26).

As such, a preferred indication in which the present invention can be applied is wound healing. The invention is especially suited to facilitate the treatment of burns, skin lesions, skin injuries or skin grafts, diabetic wounds and diabetic ulcers, e. g. diabetic foot ulcer.

Further non-limiting examples in which the present invention can be applied are the prevention or therapy of diseases associated with bone and/or cartilage damage or affecting bone and/or cartilage disease, or generally situations, in which cartilage and/or bone formation is desirable or for spinal fusion, prevention or therapy of damaged or diseased tissue associated with connective tissue including tendon and/or ligament, periodontal or dental tissue including dental implants, neural tissue including CNS tissue and neuropathological situations, tissue of the sensory system, liver, pancreas, cardiac, blood vessel, renal, uterine and thyroid tissue, mucous membranes, endothelium, epithelium, for promotion or induction of nerve growth, tissue regeneration, angiogenesis, induction of proliferation of progenitor cells and/or bone marrow cells, for maintenance of a state of proliferation or differentiation for treatment or preservation of tissue or cells for organ or tissue transplantation, for integrity of gastrointestinal lining, for treatment of disturbances in fertility, contraception or pregnancy. Diseases concerning sensory organs like the eye are also to be included in the preferred indication of the pharmaceutical composition according to the invention. As neuronal diseases again Parkinson's and Alzheimer's diseases can be mentioned as examples.

The biological activities of GDF-5-related proteins can be easily determined with the help of established test systems. Most useful and preferred is a common in vitro test known as alkaline phosphatase (ALP) assay (Takuwa et al. 1989, Am. J. Physiol. 257, E797-E803), which is also described in example 8. GDF-5-related proteins have been demonstrated to increase alkaline phosphatase activity i.e. in ROB-C26 cells (Yamaguchi et al. 1991, Calcif. Tissue Int. 49, 221-225) as described in WO95/04819, in embryonic ATDC5 cells (Riken Gene Bank, ROB 0565), in mouse stromal MCHT-1/26 cells, and in HPDL cells as shown in Nakamura et al. 2003, J. Periodontal Res. 38,597-605.

The concentrations of GDF-5-related proteins in the compositions of the invention should be chosen in dependency on the mode and period of application. Basically, GDF-5-related proteins are highly potent cytokines which are capable of eliciting effects even in exiguous quantities. As easily determinable with the help of different biological assay systems such as i.e. the alkaline phosphatase assay described herein, a concentration of 0.1 pg GDF-5 per ml of the respective solution is sufficient to cause biological actions. Accordingly, low concentrations, i.e. ranging from 0.1 pg/ml to 1 ng/ml or less, are preferred if the compositions of the invention are repeatedly administered. However, maximum effects are achievable with higher growth factor concentrations of 1 - 100 ng/ml. An independent dose response analysis of GDF-5 action utilizing a wide range of serial dilutions (0.3-80 ng/ml, Farkas et al. 1997, Neurosci. Lett. 236, 120-122) gave optimal results at a concentration of 20 ng GDF-5 per ml. In vivo skin models commonly use high doses of 1-10 μg/ml. Therefore, in a preferred embodiment of the invention, the compositions of the invention contain GDF-5 related proteins in concentrations of between 0.1 pg/ml and 10 μg/ml. Preferred total doses of GDF-5 related proteins in case of one time administrations range from 10 ng to 10 μg.

A further aspect of the invention relates to additional ingredients and components of the formulations disclosed herein.

Most administration procedures require compositions having a nearly physiological pH. Unfortunately, GDF-5 and the closely related proteins GDF-6 and GDF-7 feature poor solubility at pH values between pH 4 and pH 9. Although GDF-5-related proteins may also be administered by means of aqueous solutions having a pH around or below 4, another way to overcome the solubility problem is the adhesion to a specific colloidal drug carrier of very small particle size. The interaction between growth factor and the selected microstructured carrier efficiently prevents the undesired coagulation of the protein at slightly acid/basic and even at neutral pH. In principle, various colloidal carriers known in the art may be utilized for cosmetic and pharmaceutical compositions. Suitable carriers are extensively described in the literature (see i.e. Barrat et al. 2003: Colloidal drug carriers: achievements and perspectives. Cell. mol. life sci. 60, 21-37). Among several others, frequently used colloidal carriers are i.e. liposomes, mixed micelles, microemulsions, lipid microparticles and polymeric nanoparticles. Lipid microparticles may be further divided into lipid nanospheres (diameter below 200 nm) and micospheres (diameter 0.2-100 μm). Creation and testing of these and other colloidal drug carriers is a routine matter which can be done without undue burden.

In addition, the formulation might comprise natural and synthethic lipids. All kinds of natural and synthetic oils/lipids can be used as long as they are biocompatible, for example synthetic oils or saturated esters such as ethyl palmitate, isopropyl palmitate, alkyl myristates such as those of isopropyl, butyl and cetyl, hexyl stearate, triglycerides (i.e. of octanoic or decanoic acids, medium chained tryglycerides such as Miglyol® 812), cetyl ricinoleate, stearyl octanoate (purcelllin oil) and hydrogenated polyisobutene, or natural oils such as e.g. cottonseed, soybean, sesame, sunflower, safflower, olive, avocado, peanut, walnut, almond and hazelnut oil.

The formulation might also comprise emulsifying agents, for example phospholipids such as phosphatidylserine, phosphatidylcholine or phosphatidylethanolamine, distilled monoglycerides, mono- & diglycerides, acetic acid esters of monoglycerides, organic esters of monoglycerides, sorbitan esters of fatty acids, propylene glycol esters of fatty acids and polyglycerol esters of fatty acids.

Other bioactive protein(s) in addition to GDF-5-related proteins might also be part of the compositions of the invention. It has been shown that TGF-β increases the size of regenerated dermis and stabilizes the dermoepithelial junction (Fitzpatrick and Rosen, J. Cosmet. Laser Ther, 5: 25-34 (2003)). A cocktail (TNS Recovery Complex, SkinMedica, Inc. Carlsbad, Calif., USA) containing seven cytokines (VEGF, IL-6 and -8, HGF, PDGF-a, GCSF, and TGF-β1) derived from neonatal foreskin fibroblasts was tested in a multicenter study. Evaluation showed improvement in skin texture, and decreased wrinkling (Rokhsar, C. K. et al., Dermatol. Surg. 31: 1166-1178 (2005)). Recombinant epidermal growth factor (ReVive Skincare); and N-furfuryladenine (kinetin) plant growth factor are also on the market. All these proteins may be used together with the GDF-5-related proteins of the invention. Other proteins which act synergistically if combined with GDF-5-related proteins are disclosed in the literature/patents, i.e. in WO 99/15191. Preferred are neurotrophins, hedgehog proteins and proteins of the transforming growth factor family, including but not limited to TGF-alpha's, TGF-beta's, activins, BMP's and GDF's. Especially preferred is a combination with any one of EGF, TGF-β1, TGF-β2, TGF-β3, NGF and/or GDNF.

Other acceptable components in the compositions are:

Retinoids (vitamin A derivatives) which preserve the integrity of mucosal/epithelial surfaces;

Hydroxy acids (organic carboxylic acids further classified into alpha hydroxy acids (AHA) and beta hydroxyl acid (BHA)) which enhance epidermal shedding, i.e. glycolic acid, lactic acid, citric acid, mandelic acid, malic acid, and tartaric acid;

Antioxidants which counteract the harmful effects of free radicals, i.e. vitamin C, vitamin E, panthenol, lipoic acid, ubiquinone, niacinamide, dimethylaminoethanol, spin traps, melatonin, catalase, glutathione, superoxide dismutase, peroxidase, glucpyranosides, polyphenols, cysteine, allantoin, furfuryladenine, uric acid, and carnosine;

Depigmenting agents which alleviate hyperpigmentation, i.e. N-acetyl-4-S-cysteanimylphenol, kojic acid, arbutin, azaleic acid, paper-mulberry compound, chemical peeling agents (resorcinol, salicylic acid), Kligman's formula, Pathak's formula, and Westerhof's formula;

Botanicals, i.e. chamomile, ginseng, Gingko biloba, curcumin, glycyrrhizin, capsaicin, and aloe vera;

Glycosaminoglycans which support epidermal regeneration, i.e. hyaluronic acid;

Anticellulites which mediate lipolysis, i.e. beta-adrenergic stimulators such as theobromine, theophylline, aminophylline, caffeine, epinephrine and alphal-adrenergic stimulators such as yohimbine, piperoxane, and phentolamine;

Enzymes such as papaine and DNA repair enzymes;

Hormones, i.e. estrogens, progesterone, testosterone, and growth hormone;

Antimicrobial agents, i.e. triclosan, chlorhexidine, povidone iodine, hydrogen peroxide, antidandruff preparations, zinc pyrithione;

Chemical UV filters, i.e. 3-benzylidene camphor (3-BC) or 4-methylbenzylidene camphor (4-MBC);

Furthermore buffers, stabilizers, preservatives, reducing agents, anti-oxidant chelating agents, agents that modify isotonicity, deodorants, anaesthetics, adjuvants and solubility-enhancing additives.

These are only non-limiting examples of possible additives, and a worker skilled in the art may easily add other excipients which are currently in use which are generally regarded as safe. For more information about methods for formulating a pharmaceutical composition and selection of pharmaceutically acceptable substances please see i.e. Remington's Pharmaceutical Sciences (luth ed.; Mack Publishing Company, Eaton, Pa., 1990), Wang et al. (1980), J. Parent. Drug Assn. 34 (6): 452-462 (1980); Wang et al. (1988), J. Parent. Sci. and Tech. 42: 4-26; Lachman et al. (1968), Drug and Cosmetic Industry 102(1): 36-38, 40 and 146-148; and Akers (1988) J. Parent. Sci. and Tech. 36 (5): 222-228.

The present invention further comprises the following items:

Item 1)

A medical device composed of at least a liquid component A comprising one or more dissolved GDF-5 related proteins, and a solid component B, characterized by
a) said liquid component A comprising 0.001 to 5% of an organic additive selected from the group consisting of trehalose, dextran sulphate, carboxymethyldextran and carboxymethyldextran sulfate, thus perpetually preventing binding of at least 75% of the contained GDF-5 related protein(s) to component B and
b) said solid component B comprising one or more amorphous thermoplastic polymers.

Item 2)

The medical device according to the previous item, wherein said amorphous thermoplastic polymers are selected from the group consisting of polysulfone-based plastics and polyetherimides.

Item 3)

The medical device according to any one of the previous items, wherein said solid component B comprises one or more synthetic or semi-synthetic structural elements for storage of liquid component A and/or for delivery of liquid component A to the tissue regeneration site.

Item 4)

The medical device according to the previous item, wherein said synthetic or semi-synthetic structural element for the storage and/or delivery of liquid component A to the tissue regeneration site is selected from the group consisting of a hollow fibre, a capillary, a tubing, a tank, a container, a mesh, a spongy element, and/or a membrane.

Item 5)

Use of a medical device according to any one of items 1 to 4 for the improved healing of wounds including diabetic and other ulcers, burns, skin injuries and/or skin grafts, for the induction of nerve growth or prevention of neuronal death, for the promotion of angiogenesis, for the induction of proliferation of progenitor cells and/or bone marrow cells; for maintenance of a state of proliferation or differentiation for treatment or preservation of tissue or cells for organ or tissue transplantation; for the treatment of degenerative disorders concerning the joints to skeletal elements and/or for meniscus and/or spinal/intervertebral disk repair.

Item 6)

Use of a medical device according to any one of the previous claims for the promotion of tissue regeneration, said tissue being selected from the group consisting of skin tissue, connective tissue, bone, cartilage, connective tissue attachment, tendon, ligament, spinal/intervertebral disk, meniscus, dental tissue, dentin, periodontal ligament, hair, tissues of the sensory system, liver, pancreas, cardiac, blood vessel, renal, uterine and thyroid tissue, mucous membranes, endothelium, epithelium or neural tissue.

Item 7)

A method for the prevention of binding of GDF-5 related proteins in solution to amorphous thermoplastic polymers, biodegradable materials or polyvinylpyrrolidon, characterized by adding trehalose, dextran sulfate, carboxymethyldextran or carboxymethyldextran sulfate (final concentration: 0.001 to 5%) to said solution of GDF-5 related proteins.

Item 8)

The method according to the previous item, wherein said biodegradable materials are selected from the group consisting of woven or nonwoven collagen, gelatine, polylactide (PLA), polyglycolide (PGA), polycaprolactone (PCL) polylactid, dextran, hyaluronic acid and chitosan or combinations thereof.

The following non-limiting examples together with the figures and sequence protocols are intended to further illustrate the invention.

SEQ ID NO:1 shows the DNA sequence, and SEQ ID NO:2 shows the protein sequence of the human GDF-5 precursor.

SEQ ID NO:3 shows the DNA sequence, and SEQ ID NO:4 shows the protein sequence of the human mature monomeric GDF-5.

### FIGURES

FIG. 1 shows additional features of the human GDF-5 precursor protein according to SEQ ID NO:2: aa 001-381 pre-prodomain (bold letters)

aa 001-027 signal peptide (bold and underlined)

aa 382-501 mature protein part

aa 400-501 cystine-knot-domain (underlined)

FIG. 2 shows a comparison of the 102 aa cystine-knot domains of human GDF-5 (SEQ ID NO:2), human GDF-6 (sequence 2 from U.S. Pat. No. 5,658,882) and human GDF-7 (sequence 26 from U.S. Pat. No. 5,658,882). Amino acid residues which are identical in all three molecules are highlighted by borders.

FIG. 3 shows a table with the sequence identities of cystine-knot domains of several known BMPs and GDFs to the cysteine-knot-domain of human GDF-5.

FIG. 4 demonstrates the electrostatic charge pattern of GDF-5 dimer at neutral pH. Regions with similar charge show identical colours.

FIG. 5 shows a repeating unit of polysulfone-based plastics.

FIG. 6 shows a repeating unit of polysulfones (PSU).

FIG. 7 shows a repeating unit of polyethersulfones (PES).

FIG. 8 shows a repeating unit of polyphenylsulfones (PPSU).

FIG. 9 shows a repeating unit of polyetherimides.

FIG. 10 shows an rhGDF-5 sandwich ELISA as described in example 1. The content of rhGDF-5 dissolved in phosphate buffer saline (PBS) was compared before (black bar) and after (white bar) hollow fibre capillary passage. The rhGDF-5 content was analyzed by rhGDF-5 sandwich ELISA. After hollow fibre capillary passage no GDF-5 could be detected. Most likely the rhGDF-5 protein adhered unspecifically to the capillary walls.

FIG.11 shows an rhGDF-5 sandwich ELISA as described in example 2. The content of rhGDF-5 dissolved in 10 mM HCl, pH 2.0 was compared before (black bar) and after (white bar) hollow fibre capillary passage. The rhGDF-5 content was analyzed by rhGDF-5 sandwich ELISA. After hollow fibre capillary passage no GDF-5 could be detected.

FIG. 12 shows an rhGDF-5 sandwich ELISA as described in example 3. Hollow fibre capillaries were preincubated with human serum to block unspecific binding sites for rhGDF-5. Then 5 mL rhGDF-5 (1 μg/mL dissolved in PBS) were injected into the device. The content of rhGDF-5 was compared before (black bar) and after (white bar) hollow fibre capillary passage. The rhGDF-5 content was analyzed by rhGDF-5 sandwich ELISA. After hollow fibre capillary passage no GDF-5 could be detected.

FIG. 13 shows an rhGDF-5 sandwich ELISA as described in example 4. Hollow fibre capillaries were rinsed with 1% dextran sulfate solution. Then hollow fibres were injected with 5 mL rhGDF-5 in 1% dextran sulfate with increasing rhGDF-5 concentrations ranging from 10 to 1000 ng/mL. The rhGDF-5 content before and after hollow fibre capillary injection was determined by rhGDF-5 sandwich ELISA. The rhGDF-5 input concentration was set to 100%. rhGDF-5 could be detected in the fraction after hollow fibre capillary passage. The blocking of free binding sites with dextran sulfate did prevent the unspecific adhesion of rhGDF-5 to the capillary surface. In contrast thereto, blocking of free binding sites with serum proteins did not prevent the unspecific adhesion of rhGDF-5 to capillary surfaces.

FIG. 14 shows the results of an rhGDF-5 sandwich ELISA as described in example 4. Hollow fibre capillaries were rinsed with 0.001% dextran sulfate solution. Then hollow fibres were injected with 5 mL 1 μg/mL rhGDF-5 in 0.001% dextran sulfate. The rhGDF-5 content before and after hollow fibre capillary injection was determined by rhGDF-5 sandwich ELISA.

FIG. 15 shows the results of an rhGDF-5 sandwich ELISA as described in example 5. Hollow fibre capillaries were rinsed with 1% trehalose, 5 mM sodium acetate, pH 5. Then hollow fibres were injected with 5 mL rhGDF-5 (2 μg/mL) in 1% trehalose, 5 mM sodium acetate, pH 5. The rhGDF-5 content before and after hollow fibre capillary injection was determined by rhGDF-5 sandwich ELISA.

FIG. 16 shows the results of a stability study for rhGDF-5 at 37° C. for 1 week as described in example 6. rhGDF-5 was formulated either in 1% trehalose or 0.1% dextran sulfate. The rhGDF-5 content for the time point day 1 to day 7 (T1 to T7) was determined by rhGDF-5 sandwich ELISA. An untreated rhGDF-5 sample served as an internal control and was set to 100%.

FIG. 17: shows a freeze/thaw stability study for rhGDF-5 as described in example 7. rhGDF-5 was dissolved either in 1% trehalose or 0.1% dextran sulfate. rhGDF-5 samples were frozen at −80° C. and thawed at room temperature four times. The rhGDF-5 content for the freeze/thaw cycles (C1 to C4) was determined by rhGDF-5 sandwich ELISA. An untreated rhGDF-5 sample served as an internal control and was set to 100%.

FIG. 18 shows a dose response analysis of alkaline phosphatase (ALP) activity in MCHT1/26 cells as described in example 8. MCHT1/26 cells were stimulated with 4.8-1200 ng/mL of GDF-5 dissolved in 10 mM HCl (black triangles) and in 1% trehalose, 5 mM sodium acetate pH 5.0 (white circles) respectively. ALP activity was measured by the conversion of p-nitrophenolphosphate to p-nitrophenolate at 405 nM. The data are average values of three independent measurements.

FIG. 19 shows in vivo wound healing study on pig skin according to example 9. rhGDF-5 (150 ng/mL) formulated in 1% trehalose, 5 mM sodium acetate pH 5.0, was applied via a hollow fibre application system. In contrast to the untreated control wound (left side), the rhGDF-5 treated partial-thickness defect (right side) was regenerated within 9 days.

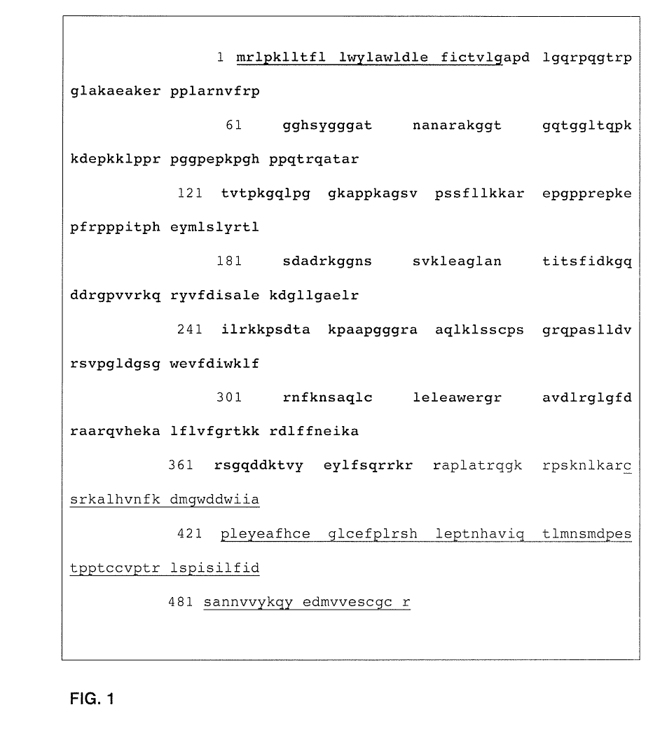

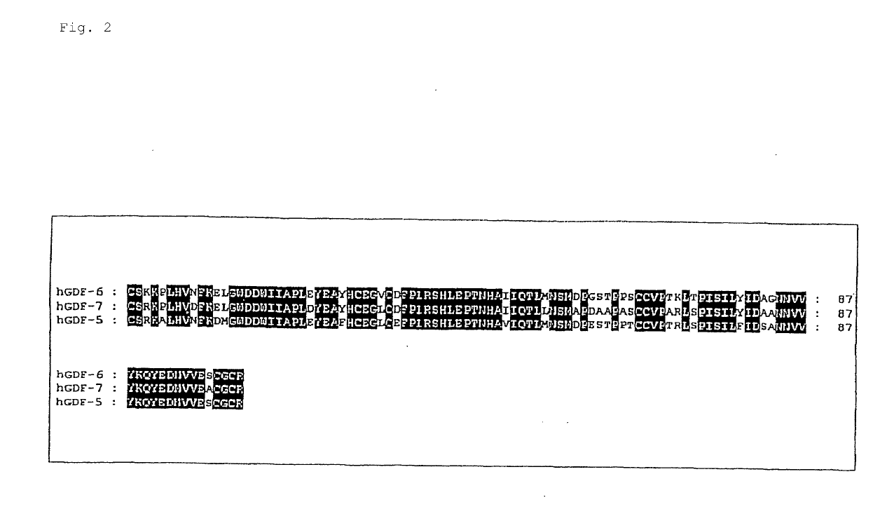

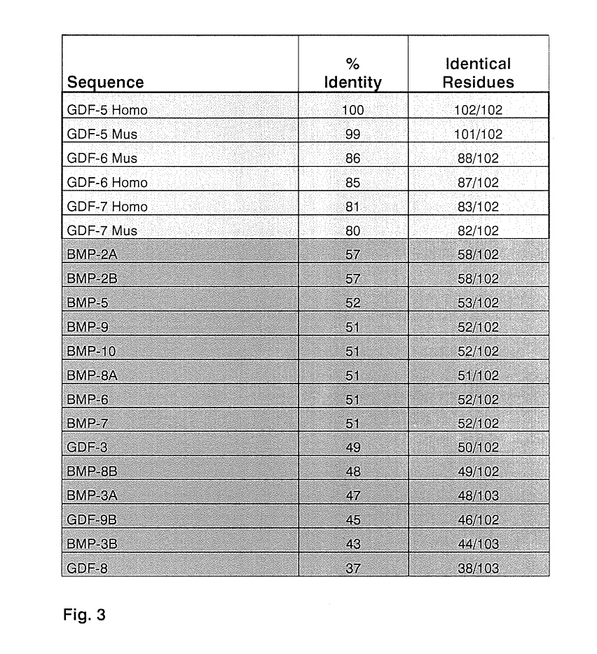

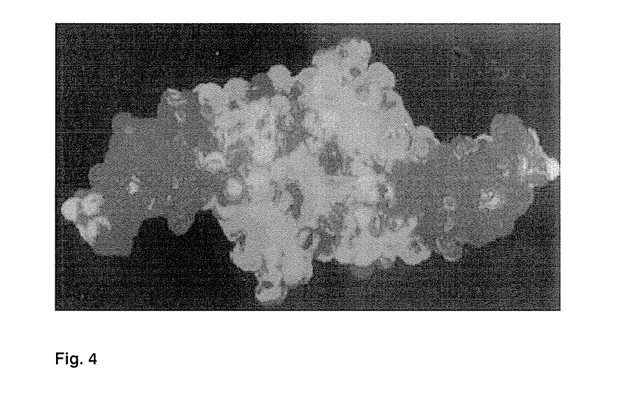

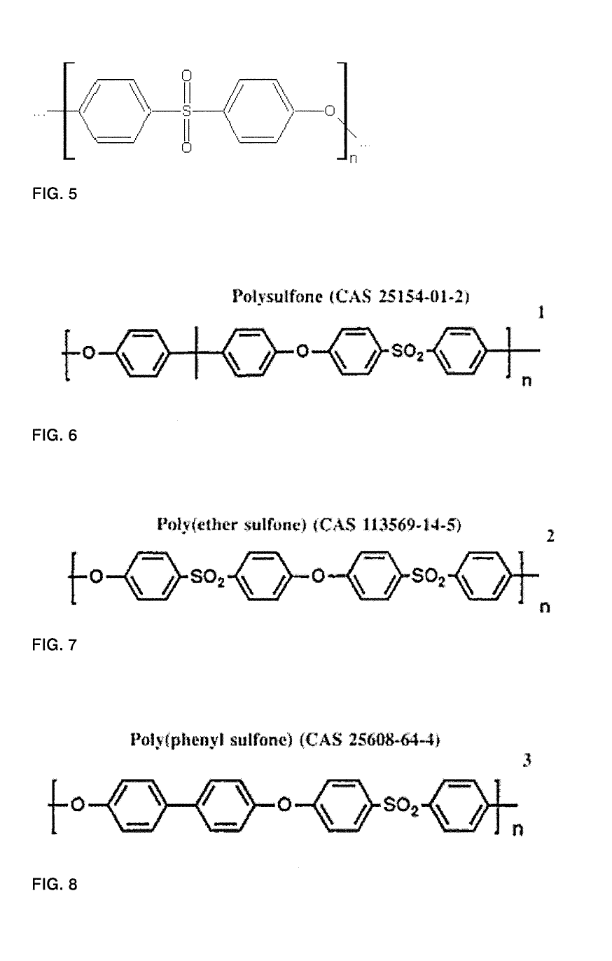

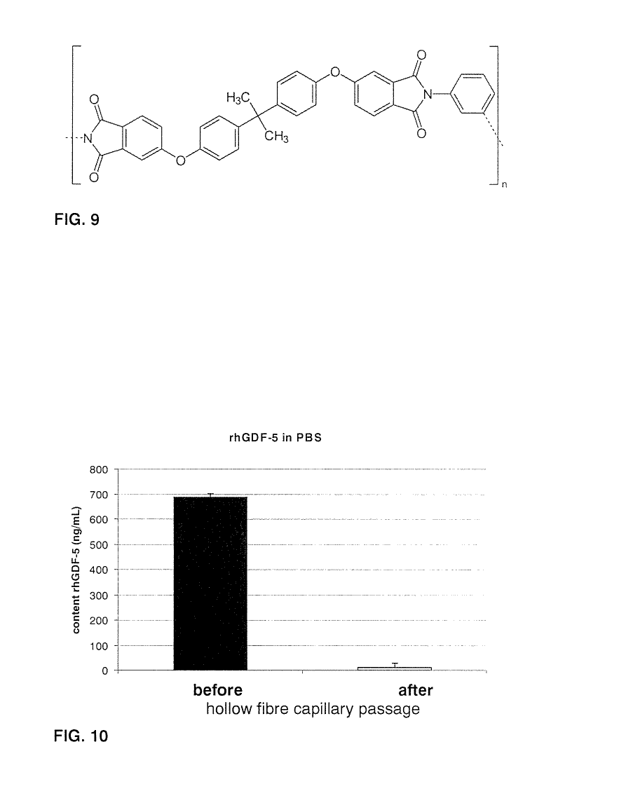

### EXAMPLES

### Example 1

### Delivery of GDF-5 Related Proteins in an Aqueous Buffer Solution at Neutral pH (PBS)

In a first set of experiments it was investigated if GDF-5 or related proteins in an aqueous buffer system pass a hollow fibre capillary device. As an example, rhGDF-5 was dissolved in lx phosphate buffered saline to generate a 6 mL rhGDF-5 solution with a final concentration of 1 μg/mL. 5 mL of the GDF-5 solution were applied with a syringe to a hollow fibre capillary device. The GDF-5 solution which left the micropores of the hollow fibre capillary device was collected in a pertri dish. To quantify the content of GDF-5 which has passed the hollow fibre capillary device, an aliquot of the sample before and after capillary passage were analyzed in a GDF-5 specific sandwich ELISA. The GDF-5 ELISA is based on two monoclonal antibodies to rhGDF-5. The enzyme avidin-peroxidase is bound to the second antibody by adding a third reagent. Detection is carried out by enzymatic conversion of the substrate tetramethylbenzidine dihydrochloride into a yellow dye, which is then determined by photometry. Unknown samples of rhGDF-5 are quantified by using a test series of rhGDF-5 standards.

In brief, the first antibody aMP-5 was adjusted to 5 μg/mL in TBS (0.01 M Tris buffered saline, 0.15 M NaCl, pH 7.4) and the ELISA plate was coated with 50 μL/well and incubated overnight at 4° C. The solution was removed and the plate was rinsed 5 times with 100 μL of rinsing solution (100 mL stock solution (0.45 M Na₂HPO₄, pH 6.5) diluted into 400 mL water p.a. and 250 μL Tween-20 was added). Next, the 50 μL/well of the standard and sample solutions were applied 4 times each, the plate was closed and incubated for 20-24 h at 4° C. The solutions were removed and the plate was rinsed 5 times with 100 μL rinsing solution. The second biotinylated antibody aMP-4 was adjusted to a concentration of 1 μg/mL with dilution buffer (10 mM TRIS, 150 mM NaCl, 0.5% Casein, 0.0004% bromophenol blue, pH 7.4), 50 μL/well were applied and the closed plate were incubated for 2 hour at 37° C. The solution was removed and the plate was rinsed 5 times with 100 μL rinsing solution. 50 μL per well of avidin-peroxidase (0.2 U/mL peroxidase activity) were added and the closed plate was incubated for 1 hour at room temperature. The solution was removed and the plate was rinsed 5 times with 100 μL rinsing solution. Next, 50 μL of the TMB (tetramethylbenzidine) dye solution was added per well and the closed plate was incubated for 30 min at room temperature. Finally, the reaction was stopped by adding 50 μL 1N H₂SO₄ per well and the absorption was measured at 450 nm.

The comparison of the rhGDF-5 content before and after passage through the hollow fibre capillary device is shown in FIG. 10. Surprisingly no rhGDF-5 could be detected in the fraction after hollow fibre capillary passage. Most likely the rhGDF-5 protein adhered unspecifically to the capillary walls.

### Example 2

### Delivery of GDF-5 Related Proteins in 10 mM HCl, pH 2.0

Since 10 mM HCl, pH 2.0, is an optimal buffer for dissolving GDF-5 related proteins, it was investigated if GDF-5 in 10 mM HCl, pH 2.0 passes a hollow fibre capillary device. The procedure was performed under the same conditions as described under example 1 with the exception that rhGDF-5 (1 μg/mL) was applied in 10 mM HCl, pH 2 instead of PBS.

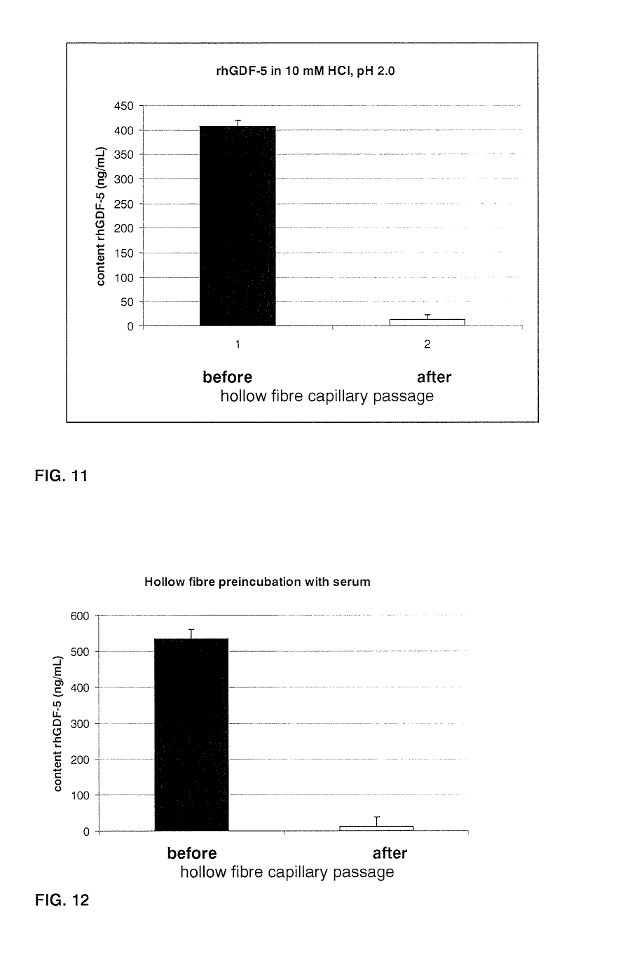

The comparison of the rhGDF-5 content, dissolved in 10 mM HCL, pH 2.0, before and after passage through the hollow fibre capillary device is shown in FIG. 11.

Surprisingly no rhGDF-5 could be detected in the fraction after hollow fibre capillary passage. Since 10 mM HCl, pH 2.0 is an optimal buffer for rhGDF-5 to achieve maximal protein solubility; it is obvious that good protein solubility is not sufficient to prevent adhesion to the capillary material. Therefore alternative strategies to reduce unspecific rhGDF-5 interaction were applied.

### Example 3

### Prevention of Unspecific Binding by Blocking with Serum Proteins

One strategy to reduce unwanted protein interaction is to block the free binding sites of the capillaries with high amounts of proteins (e.g. human serum albumin). For the blocking procedure, the hollow fibre capillary device was preincubated with 5 mL human serum. After the blocking step, 5 mL rhGDF-5 (1 μg/mL) dissolved in PBS were injected into the hollow fibre capillary device. The rhGDF-5 content before and after passage through the hollow fibre capillary device was analyzed as described in Example 1. The results are shown in FIG. 12.

rhGDF-5 could be not detected in the fraction after hollow fibre capillary passage. The blocking of free binding sites with serum proteins did not prevent the unspecific adhesion of rhGDF-5 to the capillary surface.

### Example 4

### Prevention of Unspecific rhGDF-5 Binding by Addition of Dextran Sulfate

Another strategy to reduce unwanted protein interaction is to add additives to the rhGDF-5 solution (e.g. dextran sulfate or trehalose). For this purpose rhGDF-5 was dissolved in a buffer comprising 1% (10 mg/ml) dextran sulfate (Amersham 17-0340-01, Lot 99250) in water. In this experiment the following rhGDF-5 concentrations were analyzed, 10 ng/mL, 50, ng/mL, 100 ng/mL, 500 ng/mL and 1000 ng/mL.

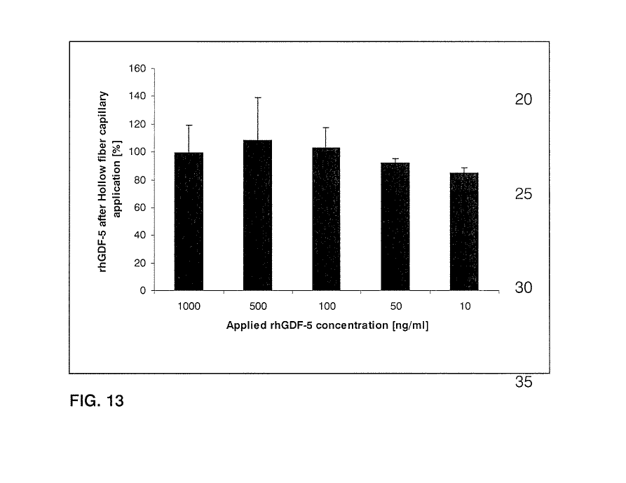

The hollow fibre capillary device was first rinsed with 5 mL 1% dextran sulfate solution without rhGDF-5. After the rinsing step 5 mL rhGDF-5 in 1% dextran sulfate solution (rhGDF-5 concentrations 10 to 1000 ng/mL) was injected separately into the hollow fibre capillary device. The rhGDF-5 content of all 5 samples before and after passage through the hollow fibre capillary device were analyzed by rhGDF-5 ELISA as described in Example 1. The results are shown in FIG. 13.

The addition of 1% dextran sulfate to rhGDF-5 a solution prevents successful the unspecific binding of rhGDF-5 to the hollow fibre capillaries. In an rhGDF-5 concentration range of 1000 to 100 ng/mL, 100% of the injected rhGDF-5 could be detected after hollow fibre capillary passage. The rhGDF-5 recovery for 50 ng/mL rhGDF-5 was 92% and for 10 ng/mL 85% respectively.

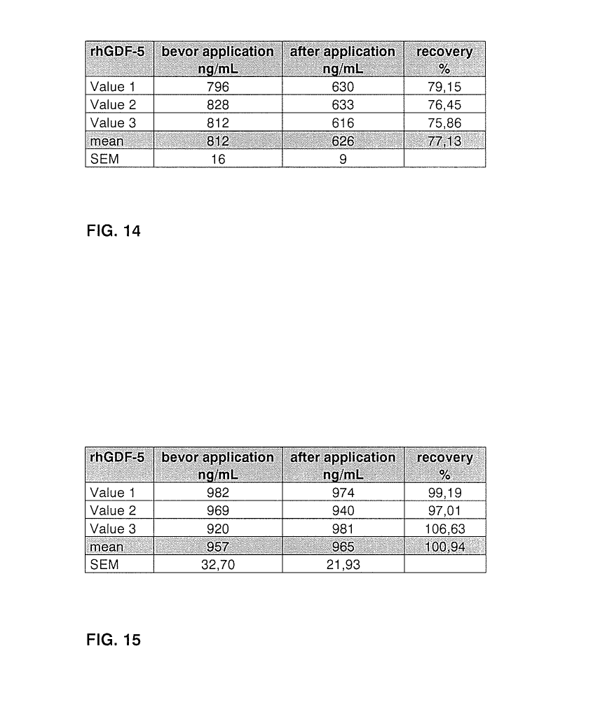

The experiments with dextran sulfate were repeated with a reduced dextran sulfate concentration (0.001% instead of 1%) and 1 μg/ mL rhGDF-5. Even with a dextran sulfate concentration of 0.001% the rhGDF-5 recovery after hollow fibre capillary passage was greater than 75% (FIG. 14).

### Example 5

### Prevention of Unspecific rhGDF-5 Binding by Addition of Trehalose

An alternative carbohydrate additive to dextran sulfate is trehalose. The experiments with trehalose were conducted as described in Example 4, with the exception that trehalose was used instead of dextran sulfate.

2 μg/mL rhGDF-5 were dissolved either in 1% or 10% trehalose, 5 mM sodium acetate pH 5.0. The hollow fibre capillary device was first rinsed with 5 mL 1% or 10% trehalose, 5 mM sodium acetate solution without rhGDF-5. After the rinsing step 5 mL rhGDF-5 (2 μg/mL) in 1% or 10% trehalose, 5 mM sodium acetate solution were injected into the hollow fibre capillary device. The rhGDF-5 content before and after passage through the hollow fibre capillary device was analyzed by rhGDF-5 ELISA as described in Example 1.

The results are shown in FIG. 15. Surprisingly no rhGDF-5 could be detected in the fraction after hollow fibre capillary passage when the rhGDF-5 solution with 10% trehalose was used. In contrast, when rhGDF-5 was dissolved in 1% trehalose, 5 mM sodium acetate, pH 5, the rhGDF-5 recovery after hollow fibre capillary passage was 100%.

### Example 6

### rhGDF-5 Temperature Stability Study with Trehalose and Dextran Sulfate

An important criteria for a wound healing drug formulation is the drug stability at body temperature. Therefore a short term stability study for rhGDF-5 in the presence of either 1% trehalose or 0.1% dextran sulfate was performed for a period of one week at 37° C. For this purpose 6 aliquots with 30 μl were prepared, containing 200 μg/mL rhGDF-5 dissolved in 1% trehalose, 5 mM sodium acetate pH 5.0 and 0.1% dextran sulfate in water respectively. The rhGDF-5 solutions were stored at 37° C. for 1 week; one aliquot was removed each day and stored at −80° C. until the stability study was finished at day 7. The rhGDF-5 content of the temperature stressed samples was compared against an rhGDF-5 aliquot which was directly stored at −80° C. without undergoing a stress test. The samples were analyzed by rhGDF-5 sandwich

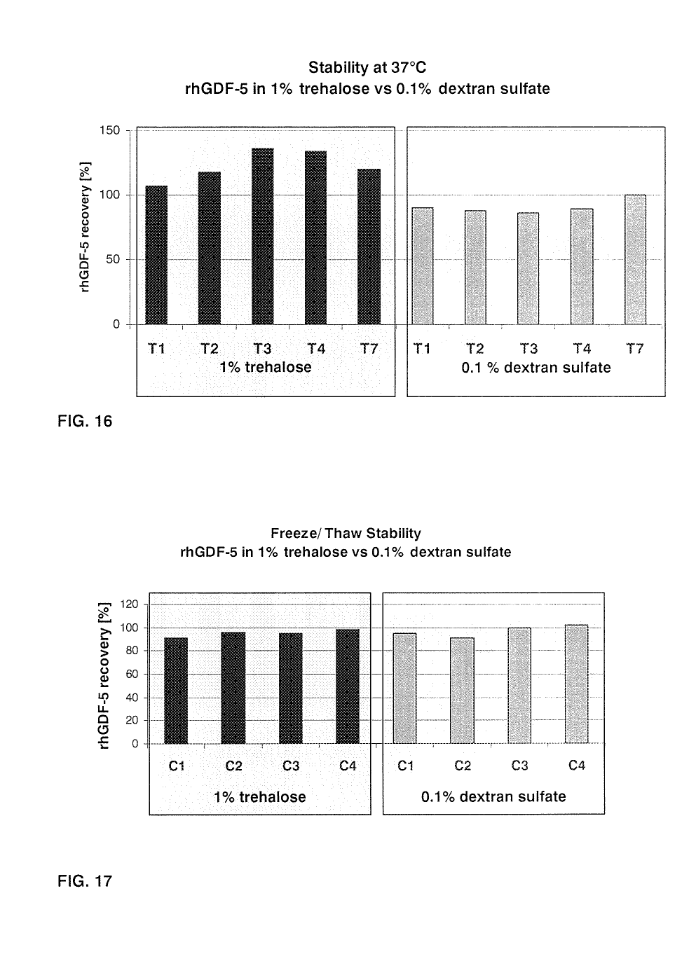

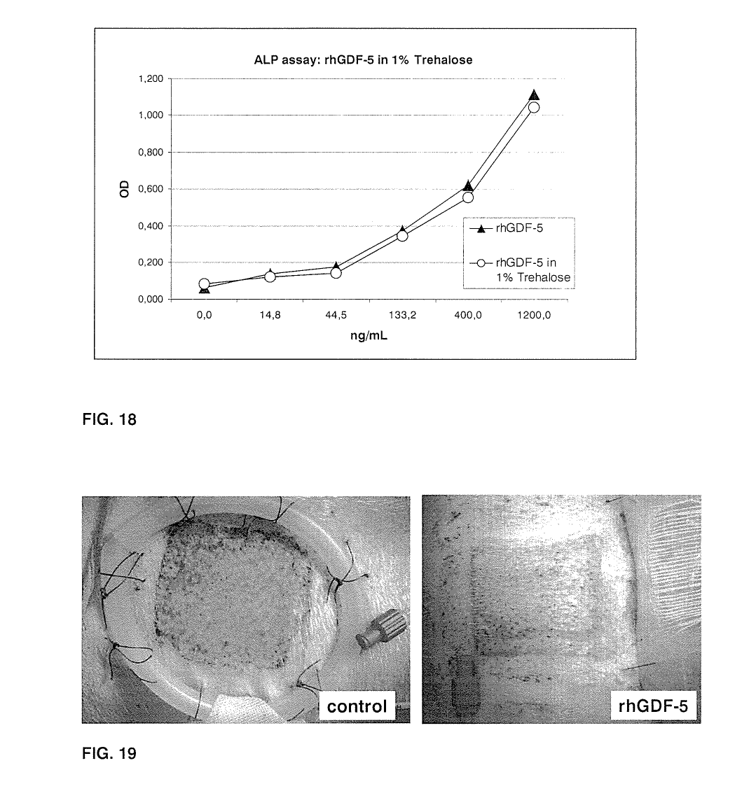

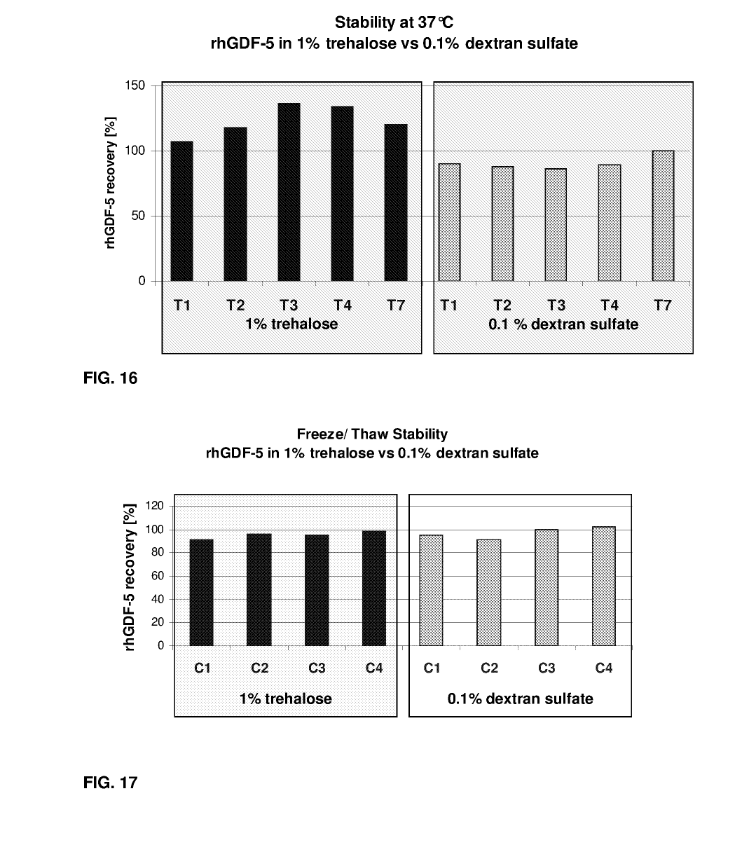

ELISA described under Example 1. The results are shown in FIG. 16. It could be demonstrated that rhGDF-5 is stable at 37° C. at least for one week, when formulated in 1% trehalose, 5 mM sodium acetate pH 5.0 or 0.1% dextran sulfate in aqueous buffers.

### Example 7

### rhGDF-5 Freeze/Thaw Stability Study with Trehalose and Dextran Sulfate

Another important aspect for drug stability is that the additives in the drug formulation supports repeated freeze/thaw cycles of rhGDF-5 without loosing bioactivity. The stability study for rhGDF-5 in the presence of either 1% trehalose or 0.1% dextran sulfate was performed for a period of one week at 37° C. For this purpose aliquots with 30 μl were prepared, containing 200 μg/mL rhGDF-5 dissolved in 1% trehalose, 5 mM sodium acetate pH 5.0 and 0.1% dextran sulfate in 1× phosphate buffered saline respectively. The rhGDF-5 solutions were stored at −80° C. over night and then thawed at room temperature. The freeze/thaw cycle was repeated four times (C1 to C4). The rhGDF-5 content of the stressed samples was compared against an rhGDF-5 aliquot which was directly stored at −80° C. without undergoing a stress test. The samples were analyzed by rhGDF-5 sandwich ELISA described under Example 1. The results are shown in FIG. 17. It could be demonstrated that rhGDF-5 could be frozen and thawed at least four times without loosing activity, when formulated in 1% trehalose, 5 mM sodium acetate pH 5.0 or 0.1% dextran sulfate in aqueous buffers.

### Example 8

### Bioactivity Study with rhGDF-5 in 1% Trehalose

To address the question if the carbohydrate additives in the rhGDF-5 formulation buffer have an influence on the GDF-5 bioactivity, a cell based alkaline phosphatase (ALP) activity assay was performed. The biological activity of rhGDF-5 (200 μg/mL) dissolved in 1% trehalose, 5 mM sodium acetate pH 5.0 was measured on mouse stromal MCHT1/26 cells (Hoechst Japan Ltd., Kawagoe, Japan). MCHT1/26 cells were plated at 4.5×10³ cells per well in 96-multiwell plates in cell culture medium (alpha-MEM, (Sigma, Taufkirchen, Germany) supplemented by 2 mM L-glutamine, (Invitrogen, Karlsruhe, Germany) and 10% fetal calf serum (Invitrogen, Karlsruhe, Germany). After 24 h, cells were stimulated with rhGDF-5 dissolved either in 10 mM HCl or in 1% trehalose, 5 mM sodium acetate pH 5.0. Prior to cell incubation, the rhGDF-5 solutions were further diluted with cell culture medium to reach rhGDF-5 concentrations ranging from 14.8-1200 ng/mL. After 72 h, cells were washed with phosphate buffered saline (PBS) and extracted with alkaline phosphate buffer 1, containing 1% Nonidet P40, 0.1 M glycine pH 9.6 (Sigma, Taufkirchen, Germany), 1 mM MgCl₂ and 1 mM ZnCl₂ (Merck, Darmstadt, Germany). To achieve thorough cell lysis, cells were incubated 15-18 h at 37° C. Alkaline phosphatase enzyme activity was assayed with 10 mM p-nitrophenylphosphate (Pierce, Bonn, Germany) as a substrate in 0.1 M glycine pH 9.6, 1 mM MgCl₂ and 1 mM ZnCl₂. After 30 min incubation at 37° C., the absorbance was measured with an automatic microplate reader (Tecan Spectra Rainbow, TECAN, Crailsheim, Germany) at 405 nM under consideration of blank value subtraction. The results are shown in FIG. 18.

rhGDF-5 was tested for its biological activity to induce ALP enzyme activation in the indicator cell line MCTH1/26 in the presence of 1% trehalose. Compared to the rhGDF-5 control sample, no influence of trehalose on the rhGDF-5 induced ALP activity could be observed. The induction of ALP activity by rhGDF-5 in the presence of 1% trehalose was dose dependently, suggesting that trehalose does not reduce the biological activity of GDF-5.

### Example 9

### Wound Healing In Vivo Study with rhGDF-5 in 1% Trehalose Applied via Hollow Fibre Capillary Device

Since wound healing is a severe problem in patients suffering from diabetes and in patients with severe burns, we have developed a dermal delivery device for rhGDF-5 to foster the healing process of chronic wounds which do not heal by themselves.

The delivery system consists of a hollow fibre network which is connected to inlet and outlet tubing, allowing the perfusion with an rhGDF-5 enriched medium. We have successfully developed a drug formulation for GDF-5, preventing the adhesion of GDF-5 to the surface of the hollow fibre walls. Since GDF-5 is a sticky protein with an unusual solubility profile, the formulation is essential for an effective rhGDF-5 delivery.

GDF-5 has a low solubility in aqueous solution at neutral pH (approx. 1 μg/mL), it is soluble within the pH ranges 2.0 to pH 4.5 and between pH 9.5 to 12.0. To reach good solubility for GDF-5, the protein should be dissolved in 10 mM HCl, pH 2.0 or in 20 mM NaAcetate buffer, pH 4.0. To investigate the influence of rhGDF-5 on cell growth, cell culture assays on isolated human cells were performed. Fibroblasts and keratinocytes were directly isolated from human skin biopsies and incubated with rhGDF-5. Interestingly, a very low concentration of rhGDF-5 (75 ng/mL) leads to an increase of cell growth of about 30%. We suggest that this effect accelerates the wound healing and regeneration process of chronic wounds (e.g. diabetic foot ulcer).

Subsequently wound healing experiments in animals with rhGDF-5 were performed. Partial thickness skin wounds (5×5 cm) were set on a pig back. Wounds were treated with rhGDF-5 (150 ng/mL rhGDF-5, 1% trehalose, 5 mM sodium acetate pH 5.0.) using a hollow fibre capillary application system. 10 mL rhGDF-5 solution were applied twice daily via the hollow fibre capillary device over a period of 9 days. The rhGDF-5 treated wounds regenerated significantly faster (FIG. 19).

1. A method for preventing the binding of GDF-5 related proteins in solution to amorphous thermoplastic polymers, biodegradable materials or polyvinylpyrrolidon, comprising adding an amount of trehalose, dextran sulfate, carboxymethyldextran or carboxymethyldextran sulfate, sufficient to prevent said binding of said GDF-5 related proteins.

2. The method according to claim 1, wherein said trehalose, dextran sulfate, carboxymethyldextran or carboxymethyldextran sulfate has a final concentration of 0.001 to 5%.

3. The method according to claim 1, wherein said trehalose is in an amount sufficient to prevent binding of at least 75% of the GDF-5 related proteins to said amorphous thermoplastic polymers, biodegradable materials or polyvinylpyrrolidon.

4. The method according to claim 3, wherein said trehalose is present in an amount between 0.001 to 3%.

5. The method according to claim 1, wherein said amorphous thermoplastic polymers, biodegradable materials or polyvinylpyrrolidon is part of a medical device.

6. The method according to claim 5, wherein said amorphous thermoplastic polymers are selected from the group consisting of polysulfone-based plastics and polyetherimides.

7. The method according to claim 5, wherein said medical device comprises one or more synthetic or semi-synthetic structural elements for storage of said GDF-5 related proteins in solution and/or for delivery of said GDF-5 related proteins in solution to the tissue regeneration site.

8. The method according to claim 7, wherein said synthetic or semi-synthetic structural elements are selected from the group consisting of a hollow fiber, a capillary, a tubing, a tank, a container, a mesh, a spongy element, and a membrane.

9. The method according to claim 1, wherein said GDF-5 related proteins are in an amount sufficient for improving the healing of wounds, for the induction of nerve growth or prevention of neuronal death, for the promotion of angiogenesis, for the induction of proliferation of progenitor cells and/or bone marrow cells; for maintenance of a state of proliferation or differentiation for treatment or preservation of tissue or cells for organ or tissue transplantation; for the treatment of degenerative disorders concerning the joints to skeletal elements and/or for meniscus and/or spinal/intervertebral disk repair.

10. The method according to claim 9, wherein said wounds are diabetic and other ulcers, burns, skin injuries and/or skin grafts.

11. The method according to claim 1, wherein said GDF-5 related proteins are in an amount sufficient for the promotion of tissue regeneration, said tissue being selected from the group consisting of skin tissue, connective tissue, bone, cartilage, connective tissue attachment, tendon, ligament, spinal/intervertebral disk, meniscus, dental tissue, dentin, periodontal ligament, hair, tissues of the sensory system, liver, pancreas, cardiac, blood vessel, renal, uterine and thyroid tissue, mucous membranes, endothelium, epithelium or neural tissue.

12. The method according to claim 1, wherein said biodegradable materials are selected from the group consisting of woven or nonwoven collagen, gelatine, polylactide (PLA), polyglycolide (PGA), polycaprolactone (PCL) polylactid, dextran, hyaluronic acid and chitosan or combinations thereof.

13. A method for preventing the binding of dissolved GDF-5 related proteins to a medical device, wherein said medical device comprises amorphous thermoplastic polymers, biodegradable materials or polyvinylpyrrolidon, comprising adding an amount of trehalose, dextran sulfate, carboxymethyldextran and/or carboxymethyldextran sulfate, sufficient to perpetually prevent binding of at least 75% of the dissolved GDF-5 related proteins to said amorphous thermoplastic polymers, biodegradable materials or polyvinylpyrrolidon, wherein said amorphous thermoplastic polymers, biodegradable materials or polyvinylpyrrolidon, are in contact with said dissolved GDF-5 related proteins during storage and/or delivery of said dissolved GDF-5 related proteins.

14. The method according to claim 13, wherein said dextran sulphate, carboxymethyldextran or carboxymethyldextran sulfate is present in an amount of 0.001 to 5%, and/or said trehalose is present in an amount of 0.001 to 3%.

15. The method according to claim 13, wherein said amorphous thermoplastic polymers are part of a hollow fiber, a capillary, a tube, a tank, a mesh, a spongy element, or a membrane.
